(19) United States
(12) Patent Application Publication
(10) Pub. No.: US 2023/0218100 A1
(43) Pub. Date: Jul. 13, 2023

(54) CONTAINER FOR RECEIVING PACKAGES

(71) Applicant: Scott Meyrowitz, Boynton Beach, FL (US)

(72) Inventor: Scott MEYROWITZ, Boynton Beach, FL (US)

(21) Appl. No.: 18/046,640

(22) Filed: Oct. 14, 2022

Related U.S. Application Data
(60) Provisional application No. 63/255,639, filed on Oct. 14, 2021.

(51) Int. Cl. A47G 29/12; A47G 29/30; A47G 29/28; A47G 29/14; A61L 2/22; A61L 2/24; A61L 2/10

(52) U.S. Cl. CPC A47G 29/1214; A47G 29/30; A47G 29/28; A47G 29/141; A61L 2/22; A61L 2/24; A61L 2/10; A61L 2202/11; A61L 2202/14; A61L 2202/122; A61L 2202/23

(57) ABSTRACT

A secure box for receiving packages is described. The secure box includes a rectangular container with a top, a bottom, a front, a left, a right, and a back. A front door forms part of the front when in a closed position, the front door having at least one hinge allowing it to move to an open position by swinging outward at a top end. A top cap with one or more photovoltaic panels formed therein, the top cap adapted to securely fasten onto the top. A package sensor senses a package placed in the rectangular container to initiate a decontamination process. A controller is electrically coupled to one or more photovoltaic panels, and the controller is communicatively coupled to the package sensor to control a decontamination system for decontaminating the package which has been sensed. The decontamination system includes at least one of ultraviolet light source, a chemical spray, or both.

### CROSS-REFERENCE TO RELATED APPLICATIONS

This application claims priority to and is a continuation in part of U.S. Provisional Patent Application 63/255,639, filed on Oct. 14, 2021, entitled “Curbside Container For Receiving Packages”, which is hereby incorporated by reference in its entirety.

### BACKGROUND OF THE INVENTION

The present invention generally relates to mailboxes and more particularly, relates to curbside boxes and containers integrated into the walls, roof, and doors for receiving packages with other features.

The delivery of packages from online retailers continues to grow. In the USA, parcel delivery volume grew over 30 percent from 2019 to 2020. This growth was in large part due to the Covid-19 pandemic. The current annual rate of parcel delivery is 3.4 billion, growing at 10-15% annually.

The present invention relates to decontaminating packages. More specifically, the present invention provides for removing or neutralizing pathogens residing on, in, or about objects placed in a mail container. Pathogens may be chemical or biological.

During September and October of 2001, terrorists sent mail contaminated with the biological agent Anthrax through the postal Service to individuals and organizations.

More recently, from late December 2019, Covid-19 virus-contaminated surfaces such as packages were delivered to homes and businesses.

Consequently, several people became ill, and several others died from exposure to this biological agent. Many people have become afraid for their own safety and the safety of their friends, associates, and loved ones. What is needed is a means for individuals or organizations to minimize their risk of illness due to exposure to contaminated mail.

### SUMMARY OF THE INVENTION

A secure box for receiving packages is described. The secure box includes a rectangular container with a top, a bottom, a front, a left, a right, and a back. The rectangular container may include insulation on all six sides to keep the packages placed inside the rectangular container cool.

A front door forms part of the front when in a closed position, the front door having at least one hinge allowing it to move to an open position by swinging outward at a top end. A top cap with one or more photovoltaic panels formed therein, the top cap adapted to securely fasten onto the top. In one example, the top cap includes a portion that is a substantially convex shape with the one or more photovoltaic panels formed therein. A package sensor senses a package placed in the rectangular container to initiate a decontamination process. A controller is electrically coupled to one or more photovoltaic panels, and the controller is communicatively coupled to the package sensor to control a decontamination system for decontaminating the package which has been sensed. The decontamination system includes at least one of ultraviolet light source, a chemical spray, or both.

The secure box may include at least one battery electrically coupled to the one or more photovoltaic panels and the controller and the decontamination system. Further, the secure box may include a doorbell button placed on the front adjacent to the front door; and a doorbell sending unit for sending a signal when the doorbell button is depressed to at least one of a door chime, a smartphone app, or both.

The secure box may include at least two cameras, each with a wide-angle lens to provide an angle of view between 90 and 180 degrees to provide a front panoramic image from the front and to provide a rear panoramic image from the back. Still, further, the secure box may include one or more lights disposed on the back to illuminate behind the secure box.

The secure box may include a wireless transceiver for providing data exchange over a local area network. Further, the secure box may include at least one wireless transceiver for providing data exchange over a local area network. A near-field transceiver adapted to sense proximity of a tag, a pin, or a bracelet coupled to an animal or human.

In another example, a secure box for receiving packages through a wall, roof, or garage is disclosed. The secure box includes a door forming part of the front when in a closed position, the front door having at least one hinge allowing it to move to an open position by swinging outward. A chute is attached to the door to create a continuous inner space for receiving packages. The secure box includes a package sensor to sense a package placed through the door to initiate a decontamination process. A controller communicatively coupled to the package sensor to control a decontamination system for decontaminating the package which has been sensed, the decontamination system includes at least one of an ultraviolet light source, a chemical spray, or both.

### BRIEF DESCRIPTION OF THE DRAWINGS

The accompanying figures wherein reference numerals refer to identical or functionally similar elements throughout the separate views, and which together with the detailed description below are incorporated in and form part of the specification, serve to further illustrate various embodiments and to explain various principles and advantages all in accordance with the present invention, in which:

FIG. 1 is a front view of the curbside container, according to one example of the present invention;

FIG. 2 is an interior view of the curbside container of FIG. 1, according to one example of the present invention;

FIG. 3 is an interior view of the curbside container of FIG. 1 with aerosol disinfectant, according to one example of the present invention;

FIG. 4 is an interior view of the curbside container of FIG. 1 with aerosol disinfectant and/or UV light, according to one example of the present invention;

FIG. 5 is an interior view of the curbside container of FIG. 1 with aerosol disinfectant, according to one example of the present invention;

FIG. 6 is a side view of the curbside container of FIG. 1 illustrating the removable top portion, according to one example of the present invention;

FIG. 7 is an interior view of the curbside container of FIG. 1 illustrating the removable top portion, according to one example of the present invention;

FIG. 8 is a rear view of the curbside container of FIG. 1 illustrating other optional features, according to one example of the present invention;

FIG. 9 is a front view of the wall or garage door mountable container illustrating other optional features, according to another example of the present invention;

FIG. 10 is a side view of the wall or garage door mountable container of FIG. 9, according to another example of the present invention;

FIG. 11 is a front view of an inside wall and an outside wall mounting flanges for the wall or garage door mountable container of FIG. 9, according to another example of the present invention;

FIG. 12 is an alternate embodiment of the wall or garage door mountable container of FIG. 9, with a drone drop chute or a roof mount chute, according to another example of the present invention;

FIG. 13 is a more detailed view of the roof mount chute of FIG. 12, according to another example of the present invention;

FIG. 14 is another more detailed view of the roof mount chute of FIG. 12, according to another example of the present invention;

FIG. 15 is a block diagram of a microprocessor-based system that implements many of the features according to one example of the present invention;

FIG. 16 is a flow diagram of the cleaning process being executed on the microprocessor system of FIG. 15, according to one example of the present invention; and

FIG. 17 is a flow diagram of the tracking tags, pins, and collars being worn by children and animals, being executed on the microprocessor system of FIG. 15, according to one example of the present invention.

### DETAILED DESCRIPTION

As required, detailed embodiments of the present invention are disclosed herein; however, it is to be understood that the disclosed embodiments are merely examples of the invention, which can be embodied in various forms. Therefore, specific structural and functional details disclosed herein are not to be interpreted as limiting but merely as a basis for the claims and as a representative basis for teaching one skilled in the art to variously employ the present invention in virtually any appropriately detailed structure and function. Further, the terms and phrases used herein are not intended to be limiting; but rather to provide an understandable description of the invention.

Any references to physical dimensions or sizes are examples only and other sizes and dimensions are within the true scope of the invention.

### Non-Limiting Definitions

Any references to physical dimensions or sizes are examples only and other sizes and dimensions are within the true scope of the invention.

The terms “a,” “an,” and “the” are intended to include the plural forms as well unless the context clearly indicates otherwise.

The phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N, that is to say, any combination of one or more of the elements A, B, . . . or N including any one element alone or in combination with one or more of the other elements which may also include, in combination, additional elements not listed.

The term “camera” as used herein, captures photographs and video in digital memory and includes visible and infra-red cameras.

The terms “comprises” and/or “comprising,” when used in this specification, specify the presence of stated features, steps, operations, elements, and/or components but do not preclude the presence or addition of one or more other features, integers, steps, operations, elements, components, and/or groups thereof.

The term “configured to” describes the hardware, software, or a combination of hardware and software that is adapted to, set up, arranged, built, composed, constructed, designed, or that has any combination of these characteristics to carry out a given function. The term “adapted to” describes the hardware, software, or a combination of hardware and software that is capable of, able to accommodate, to make, or that is suitable to carry out a given function.

The term “coupled,” as used herein, is defined as “connected,” although not necessarily directly and not necessarily mechanically.

The term “doorbell” as used herein, is a signaling device that is wired or wireless connected to a bell or smartphone app to alert delivery of a package.

The terms “including” and “having,” as used herein, are defined as comprising (i.e., open language).

The term “light” refers to a lower power consumption light technology, such as LED light, UV light, UVC light, other germicidal light sanitizer light, or a combination thereof.

### Overall Structure

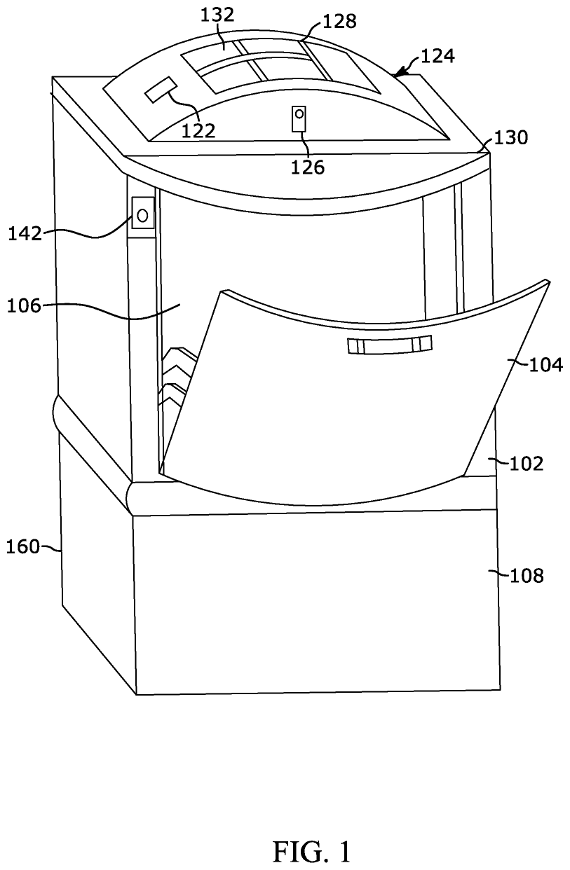

Turning now to FIG. 1 is a front view of the curbside container 100, according to one example of the present invention. Shown is a substantially rectangular container with a top 120, a bottom 108, a front 102, a left 140, a right 150, and a back 160 as shown. A modular top cap 130 is affixed to the top 120 as shown and will be described in further detail below. The bottom portion extends below the ground to provide stability and to make unauthorized relocation of the curbside container 100 more difficult.

The curbside container 100 can be fabricated from any weatherproof material, including metals, such as aluminum, plastics, composites or a combination. The outside of the curbside container can be powder coated or wrapped in a plastic film to mimic designs such as brick.

A front door 102 includes one or more hinges (not shown) forms part of the front when in a closed position. In the open position, packages and other items can be placed inside 106 the curbside container. In one embodiment, the container is not designed to replace US Postal Service mailboxes. Therefore, no US Postal requirements needed to be maintained. In another embodiment, the curbside container can be designed to meet US Postal requirements. To meet the US Postal requirement, a separate box, slot or mailbox may be formed.

The container 100 can be insulated to keep refrigerated items cool. Insulation can be batt insulation, spray foam insulation, cladding insulation, or a combination thereof.

The approximate dimensions of the box are shown as six (6) feet high with two (2) feet inserted into the ground and four (4) feet wide, and two (2) feet and two (3) feet deep. It is important to note that other dimensions are within the true scope and spirit of the present invention.

The modular top cap 130 includes one or more photovoltaic panels 132 formed therein, and the top cover is adapted to securely fasten onto the top 120 of the rectangular container 100. In one example, the modular top cap 130 includes a portion that is a substantially convex shape with one or more photovoltaic panels 132 formed therein. The modular top cap 120 is designed to be removable and replaceable, as shown below. In addition, electrical connectors, such as multiconductor plugs and sockets that are keyed, allow the top to be removed and the photovoltaic panel 132 and front camera 122, left camera 124, right camera 126, and back camera 128 (not shown).

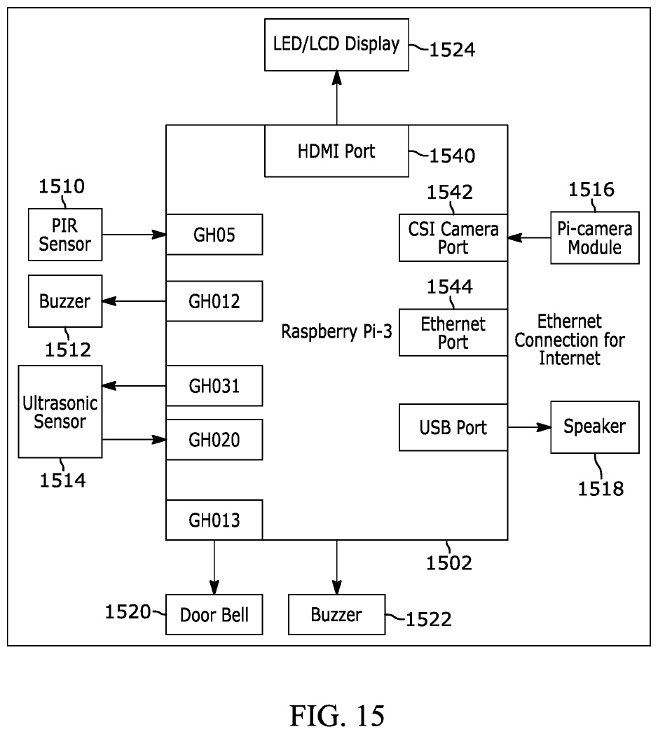

An electronic controller, such as that described below in FIG. 15, is electronically coupled to a sensor to sense a package placed in the rectangular container to initiate a decontamination process. The sensor can be an ultrasonic sensor, a passive infrared sensor, a tactile sensor, a load sensor, or any other sensor to sense the presence of a package inside the rectangular container. The controller is electrically coupled to the photovoltaic or solar panels 132, and the controller is communicatively coupled to the package sensor to control a decontamination system to start a decontamination process for decontaminating the package which has been sensed decontamination system including at least one of an ultraviolet light source, a chemical spray, or both.

Also shown is a doorbell 142 in which the delivery driver can notify the recipient of the package that it has been delivered. One or more cameras 122, 124, 126, and 128 are integrated into the top cap to provide up to a 360-degree angle of view. In one example, the camera on top of the unit goes directly to the cloud via a wireless internet connection such as Wi-Fi. In order to save power, the camera can be motion-activated or be available to stream continuously. A smartphone app allows a user to view a live stream from the camera. Optionally, the images from the camera can be recorded in a cloud-based arrangement similar to the Ring camera.

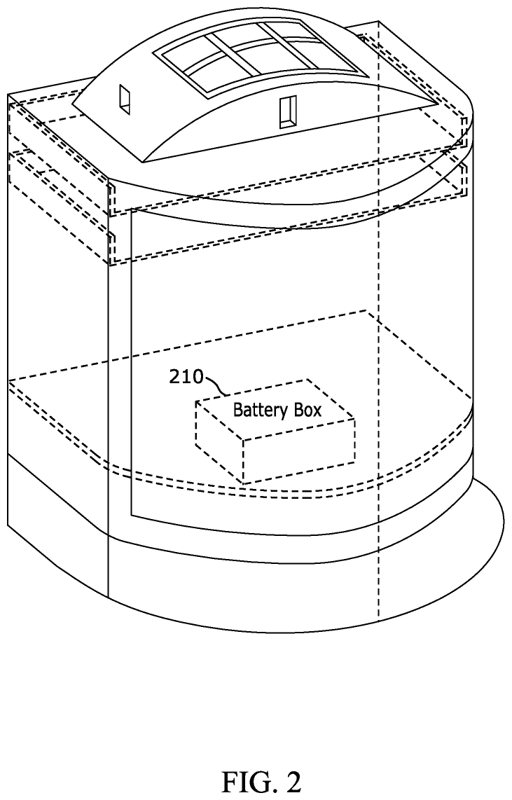

FIG. 2 is an interior view of the curbside container of FIG. 1, according to one example of the present invention;

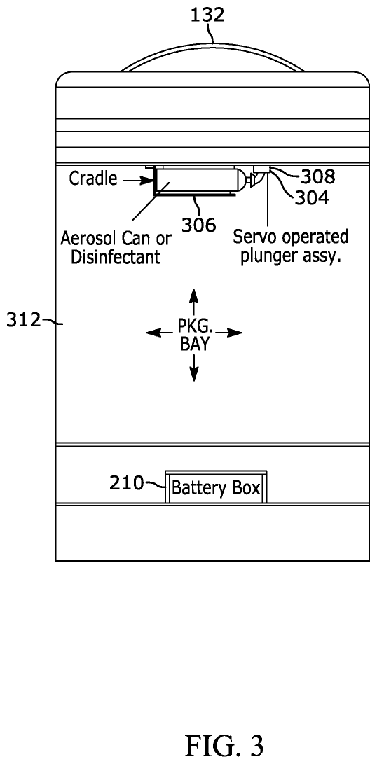

Turning now to FIG. 3 shown is an interior view of the curbside container of FIG. 1 with aerosol disinfectant, according to one example of the present invention. In this embodiment, the servo-operated to depress a plunger on an aerosol can of disinfectant.

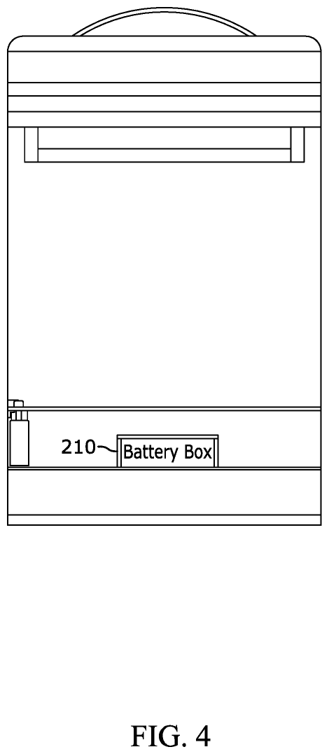

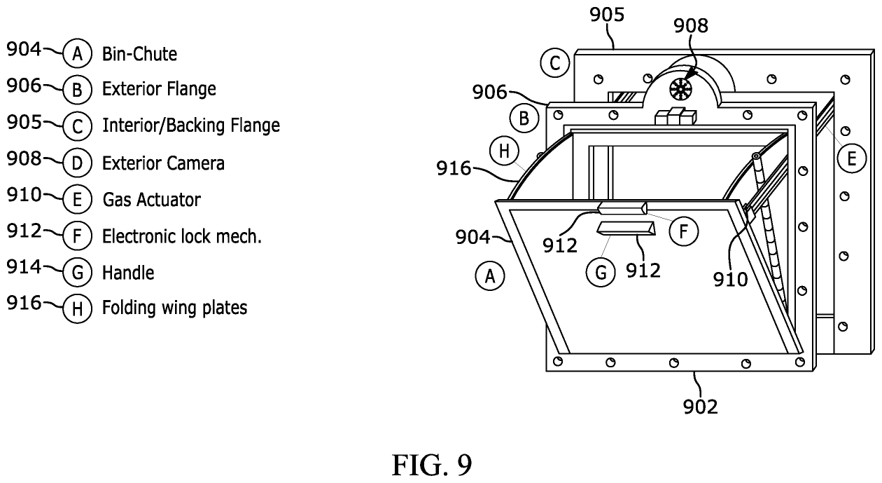

Turning now to FIG. 4 is an interior view of the curbside container of FIG. 1 with aerosol disinfectant and/or UV light, such as a UV-C light that produces radiation to disinfect air and surfaces, according to one example of the present invention. In this example, the controller in FIG. 9 will turn on the UV light for a period of time after sensing a package is placed in the rectangular container. In this embodiment, a series of tubes direct the disinfectant from the aerosol can to the interior of the container.

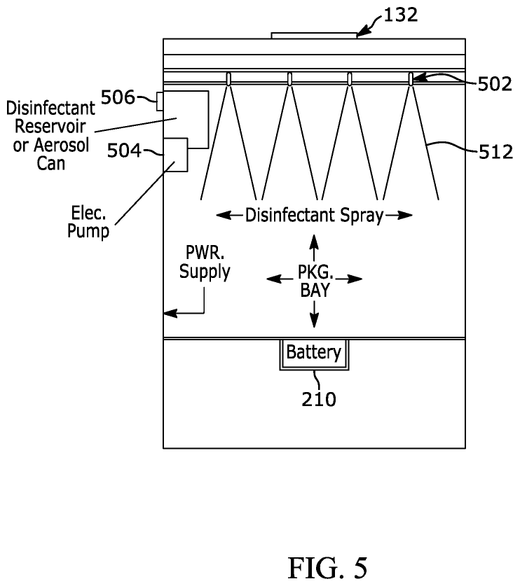

FIG. 5 is an interior view of the curbside container of FIG. 1 with aerosol disinfectant, according to one example of the present invention. In this embodiment, a series of tubes direct the disinfectant from the aerosol can to the interior of the container.

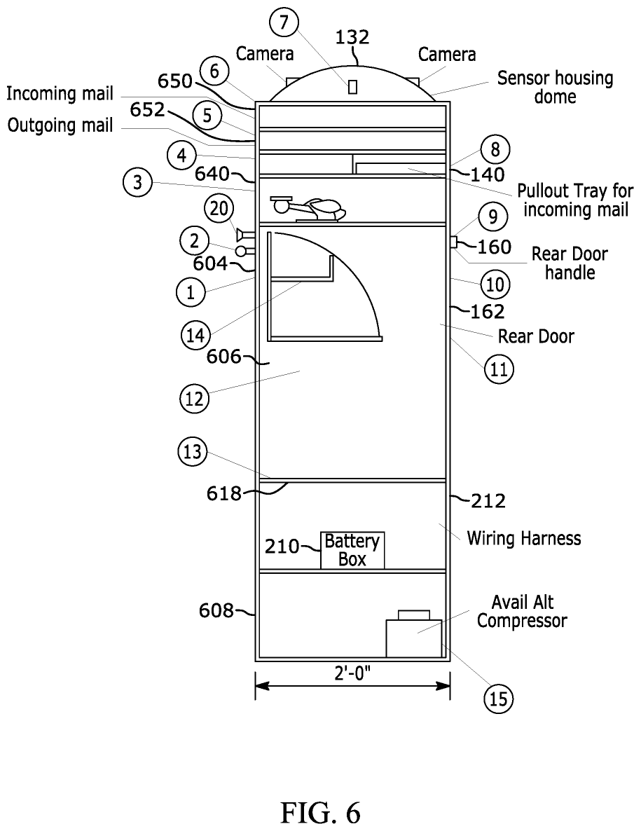

FIG. 6 is a side interior view of the curbside container of FIG. 1 with aerosol disinfectant, according to one example of the present invention. In this embodiment, a door with an optional hook for backpacks and clothing is shown. The door is made to be semicircular in profile shape, like many USPS mailboxes, to reduce theft.

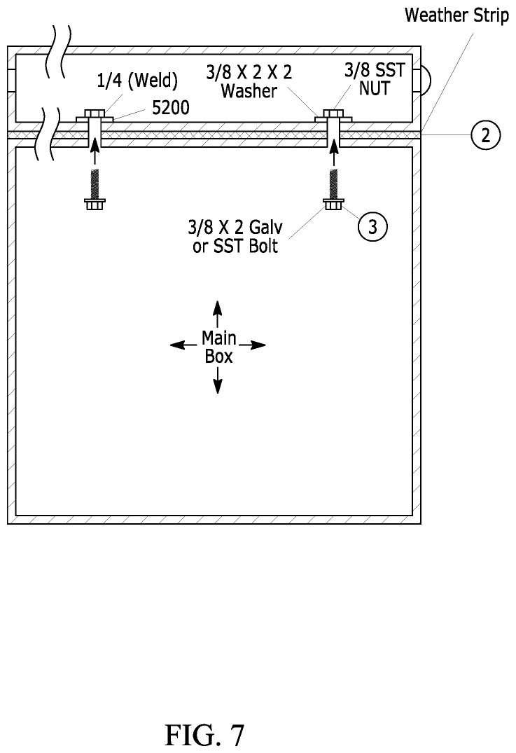

FIG. 7 is an interior view of the curbside container of FIG. 1, illustrating the removable top portion, according to one example of the present invention. Fasteners such as bolts and electrical connectors make the top modular. The control circuitry can be included at the top so that most of the intelligence is replaceable with the top. Only the battery may be left near the bottom of the rectangular container to provide stability.

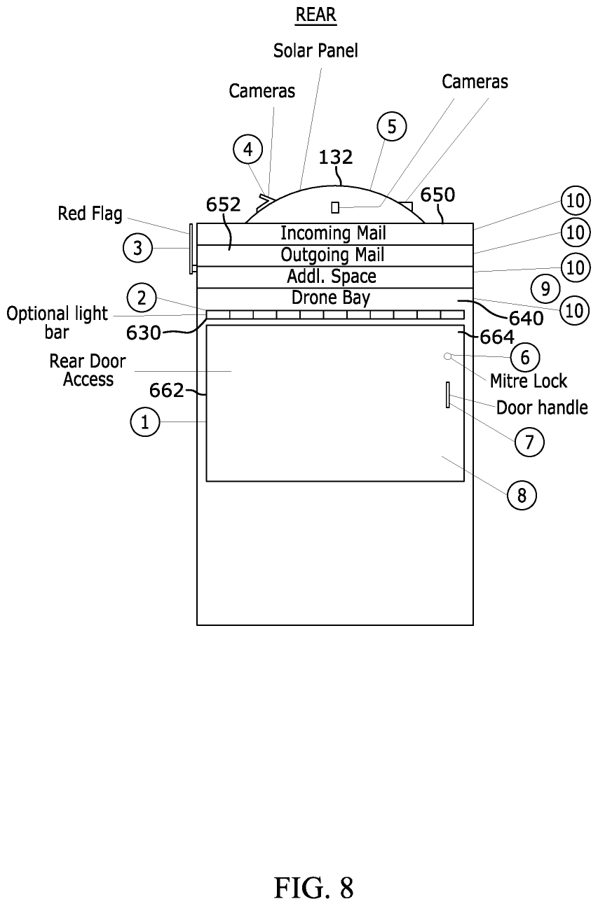

Turning now to FIG. 8 is a rear view of the curbside container of FIG. 1 illustrating other optional features, according to one example of the present invention. In this embodiment, a rear access door forms part of the rear when closed. A light bar may be affixed to the back to illuminate behind the box, including the house. Control can be made through a wireless connection back to an app running on a smartphone or in the cloud. Lens, color filters, and adapters may also be used for decoration and projects with lights during holidays. A bay for drone delivery is also shown. Drones in the rear of the unit are to be called upon to do property surveillance for security purposes. Drones inside on the top of the unit are security drones that, when called, will, in real-time, fly around the property for surveillance and security. Security for the rear door can be via a Mitre lock or similar mechanical lock. In another example, the door is accessed through a smartphone app that controls an actuator.

### Wall or Garage Mount

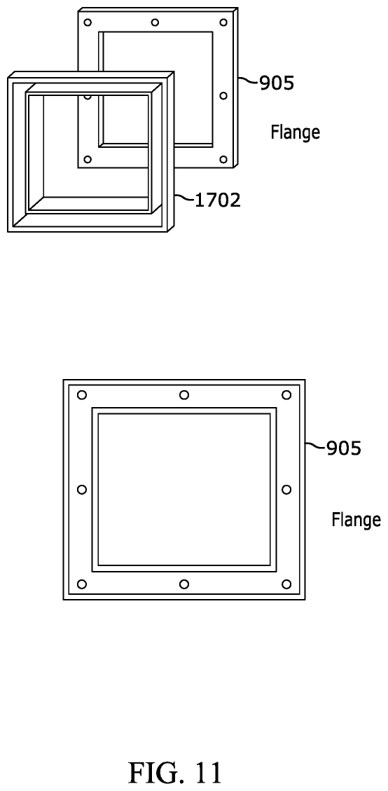

Turning to FIG. 9 is a front view of the wall or garage door mountable container 902 illustrating other optional features, according to another example of the present invention. The mountable container 902 has a flange 906 that mechanically mounts to an outside wall flange 905, as shown in FIG. 11, further described below. The mountable container includes a bin or chute drawer 904 for receiving packages. An optional camera 908 provides monitoring of the use of the mountable container 902. Additional cameras may be added to provide views of the street and surroundings depending on the position of the mountable container. In another embodiment, other cameras may be networked to provide other exterior and interior view.

The chute drawer 904 has an actuator 910, such as a gas or electronic actuator, that works in cooperation with an electronic locking mechanism 912 to open the chute to receive packages. A handle 914 and wing plate 916 to form the right and left side of the chute drawer 904, as shown. The dimensions of the mountable container 902 can be any size, and standard sizes available include 24″ by 24″, 30″ by 30″, 36″ by 30″, 36″ by 36″, 48″ by 30″, and more

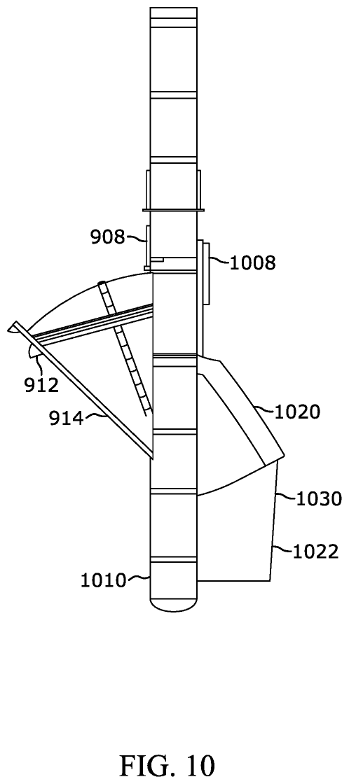

FIG. 10 is a side view of the wall or garage door mountable container of FIG. 9, according to another example of the present invention. A substantially rectangular opening garage door or wall 1010 or garage door accepts the inside wall mounting flange 1702 and the outside wall mounting flange 905 shown in FIG. 11. Also shown is an optional inside camera 1008 for monitoring the package system. Once the package is placed in chute drawer 904, it slides down into the interior sections 1020, 1022 of the chute. These interior sections can be fabricated from metal, plastic, or composite and formed as a solid wall or mesh. Like the curbside embodiment described above, a sensor detects the presence of the package and begins the disinfection process as described above.

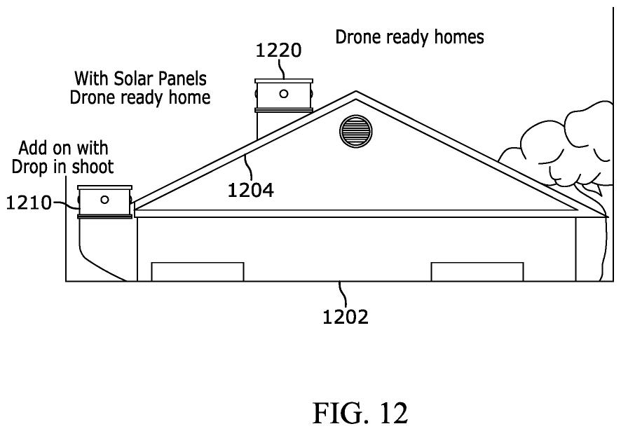

FIG. 12 is an alternate embodiment of the wall or garage door mountable container of FIG. 9. Illustrated a drone drop chute 1210 next to a house that mounts to the wall mountable chute shown in FIG. 9-11 above. Also shown is a roof mount chute 1220 to a roof 1204 of house 1202, according to another example of the present invention.

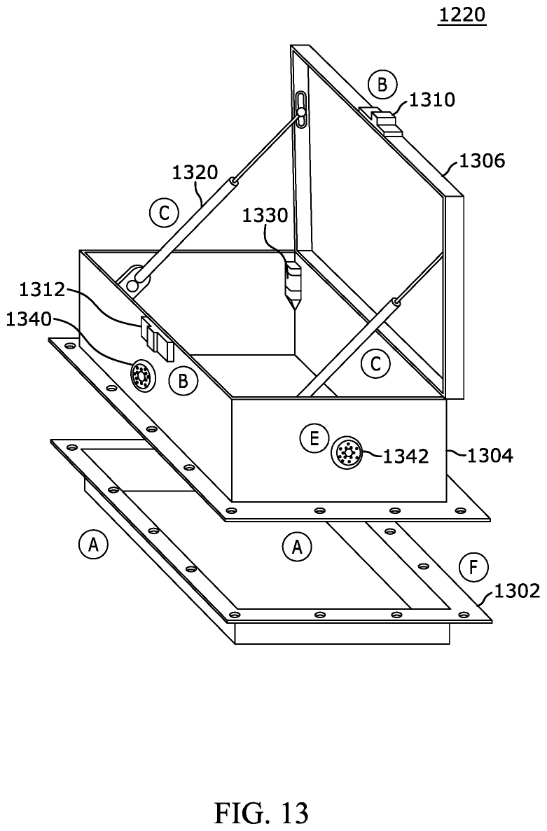

FIG. 13 is a more detailed view of the roof mount chute 1220 of FIG. 12. Shown is a roof mount flange 1302, similar to the flanges in FIG. 11 for the wall mount. A substantially rectangular box 1304 with a hinged lid 1306 is shown. When the drone is ready to deliver that package, an electronic lock 1310 is open, and the hinged lid 1306 moves to an open position as shown. The proximity of the drone may be determined through messaging, near-field communications, or recognition using one or more of the cameras 1340 as shown. An optional motion sensor 1330 is also shown. The motion sensor determines whether the package being delivered via a drone is passing through the roof mount chute 1220 to an internal chute. Once the package is in the chute, the disinfection process begins as described above for the curbside mailbox.

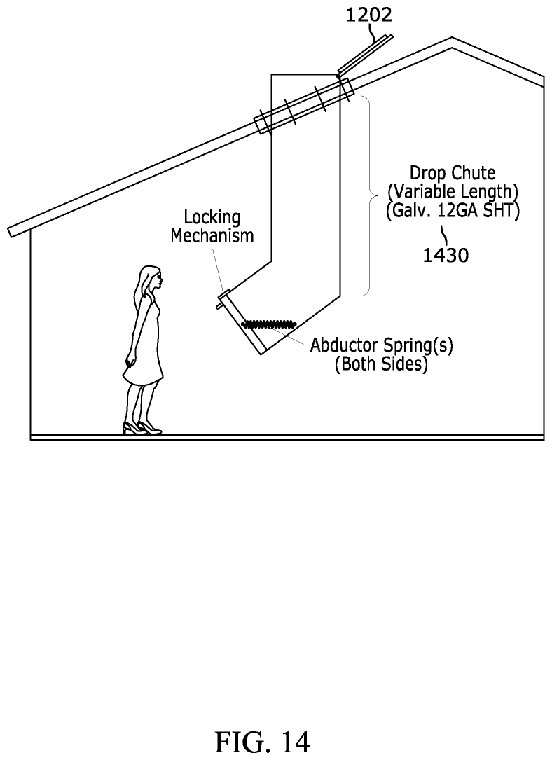

Turning to FIG. 14 is another more detailed view of the roof mount chute of FIG. 12, according to another example of the present invention. Shown are the portions of the interior chute 1430, which extends downward from the roof to a room, such as a garage or utility room to form a continuous inner space for receiving packages. The chute 1430 can have various portions with interior compartments and doors. Using dividers to form interior compartments permits packages that have been disinfected to be separate from packages going through disinfection.

### Circuitry

FIG. 15 is a block diagram of circuitry used for carrying out many of the features of the present invention. In this example, a microprocessor-based system such as Raspberry Pi 1502 is used. It performs signal fetching through sensors and processing the signal, and sending email after processing. It fetches the signal from PIR sensor 1510, cameras and microphone 16 and ultrasonic sensors 1514 and sends capture images to homeowner/registered members via email services. USB ports 1530 and GPIO (general purpose input/ouput) pins 1532, 1534, 1536, 1538, 1540 are available as connectors for connecting the sensors and devices. FIG. 15 shows various components (HDMI port 1540, Camera Serial Interface (CSI) Port 1542, Ethernet Port 1544 present on the Raspberry Pi-3 module. A battery shown above in FIG. 2 that is rechargeable by the photovoltaic solar panels is used to power the circuitry of FIG. 15. Inputs to this diagram include package pressure sensors, doorbell, cameras, near field readers to track tags, pins, collars worn by children and pets. Outputs include actuators for the UV lighting and chemical spray, door interlock, lights, and sirens.

### Flow Diagram

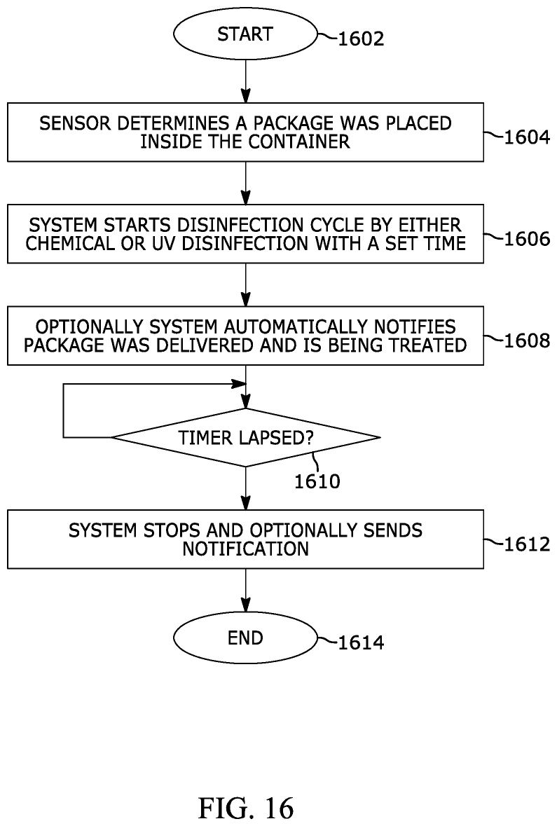

FIG. 16 is a flow diagram of the cleaning process being executed on the microprocessor system of FIG. 15, according to one example of the present invention. When the box opens and closes the client will be notified on when there packages get dropped off date, time and a picture of who delivered it. The package will immediately be disinfected by either UVC or Disinfectant spray.

The present invention unit disinfects packages with either UVC or a spray Disinfectant, Killing 99% of viruses Before the packages enter the home. This prevents outside bacteria, viruses in homes and businesses from delivery persons. Many hands touch these packages. The Covid lives for 24 to 48 hours on cardboard and on plastic anywhere from 3 to 5 days.

Through a combination of sensors, any tampering of the box a siren or alarm goes off and alerts the client via a smartphone app.

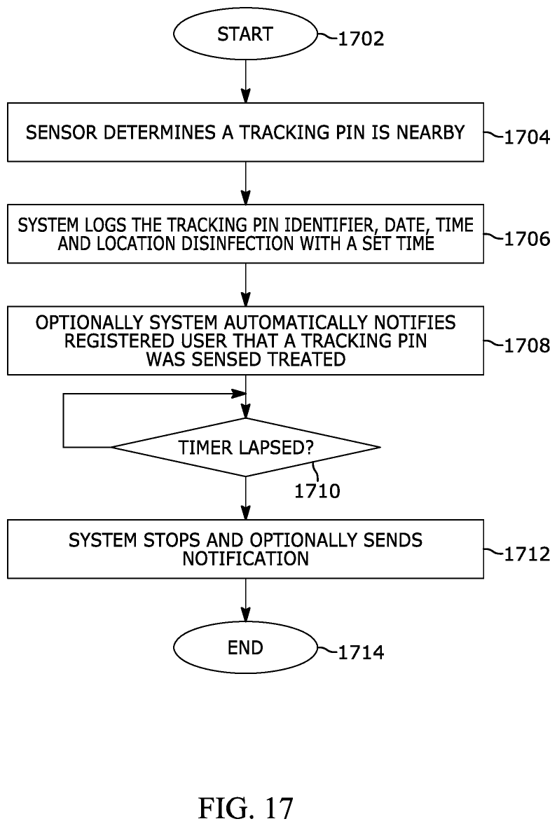

FIG. 17 is a flow diagram of the tracking tags, pins, collars being worn by children and animals, according to one example of the present invention. This has the ability to protect and track children and pets through use of a bracelet or pin or collar worn by the child or pet.

### Non-Limiting Examples

The flowchart and block diagrams in FIG. 15 through FIG. 17 illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The description of the present application has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand various embodiments of the present invention, with various modifications as are suited to the particular use contemplated.

1. A secure box for receiving packages comprising:
a rectangular container with a top, a bottom, a front, a left, a right, and a back;
a front door forming part of the front when in a closed position, the front door having at least one hinge allowing it to move to an open position by swinging outward at a top end;
a top cap with one or more photovoltaic panels formed therein, the top cap adapted to securely fasten onto the top;
a package sensor to sense a package placed in the rectangular container to initial a decontamination process; and
a controller electrically coupled to the one or more photovoltaic panels and the controller communicatively coupled to the package sensor to control a decontamination system for decontaminating the package which has been sensed, the decontamination system includes at least one of an ultraviolet light source, a chemical spray, or both.

2. The secure box of claim 1, wherein the top cap includes a portion that is a substantially convex shape with the one or more photovoltaic panels formed therein.

3. The secure box of claim 1, further comprising:
at least one battery electrically coupled to the one or more photovoltaic panels and the controller and the decontamination system.

4. The secure box of claim 1, further comprising:
a doorbell button placed on the front adjacent to the front door; and
a doorbell sending unit for sending a signal when the doorbell button is depressed to at least one of a door chime, a smartphone app, or both.

5. The secure box of claim 1, further comprising:
at least two cameras, each with a wide-angle lens to provide an angle of view between 90 and 180 degrees to provide a front panoramic image from the front and to provide a rear panoramic image from the back.

6. The secure box of claim 1, further comprising:
one or more lights disposed on the back to illuminate behind the secure box.

7. The secure box of claim 1, further comprising:
at least one wireless transceiver for providing data exchange over a local area network.

8. The secure box of claim 7, further comprising:
a near-field transceiver adapted to sense proximity of a tag, a pin, or a bracelet coupled to an animal or human.

9. The secure box of claim 1, wherein the rectangular container is insulated on all six sides to keep the packages placed inside the rectangular container cool.

10. A secure box for receiving packages through a wall, roof, or garage door comprising:
a door forming part of the front when in a closed position, the front door having at least one hinge allowing it to move to an open position by swinging outward;
a chute attached to the door to create a continuous inner space for receiving packages;
a package sensor to sense a package placed through the door to initiate a decontamination process; and
a controller communicatively coupled to the package sensor to control a decontamination system for decontaminating the package which has been sensed, the decontamination system includes at least one of an ultraviolet light source, a chemical spray, or both.

11. The secure box of claim 10, further comprising:
a doorbell button placed on the front adjacent to the front door; and
a doorbell sending unit for sending a signal when the doorbell button is depressed to at least one of a door chime, a smartphone app, or both.

12. The secure box of claim 10, further comprising:
at least one camera with a wide-angle lens to provide an angle of view between 90 and 180 degrees to provide a front panoramic image from the front.

13. The secure box of claim 12, further comprising:
at least one camera with a wide-angle lens to provide an angle of view between 90 and 180 degrees to provide a view within the chute of packages placed in the secure box.

14. The secure box of claim 10, further comprising:
at least one wireless transceiver for providing data exchange over a local area network.

15. The secure box of claim 14, further comprising:
a near-field transceiver adapted to sense proximity of a tag, a pin, or a bracelet coupled to an animal or human.
